(19) United States
(12) Patent Application Publication
(10) Pub. No.: US 2017/0003265 A1
(43) Pub. Date: Jan. 5, 2017

(54) SENSOR DEVICE

(71) Applicant: AISAN KOGYO KABUSHIKI KAISHA, Obu-shi (JP)

(72) Inventor: Nobuhiro KATO, Tokai-shi (JP)

(21) Appl. No.: 15/039,942

(22) PCT Filed: Sep. 12, 2014
(86) PCT No.: PCT/JP2014/074240

§ 371 Date: May 27, 2016

(30) Foreign Application Priority Data
Dec. 9, 2013 (JP) 2013-254307

(51) Int. Cl. G01N 33/28; F04F 5/00; G01N 27/22; F02M 37/00

(52) U.S. Cl. CPC G01N 33/2852; F02M 37/0076; F04F 5/00; G01N 27/221; B60K 2015/0321

(57) ABSTRACT

A sensor device 1 includes a fuel property sensor 5 configured capable of detecting a property of fuel that is ejected from a fuel pump 4 in a feel tank 2; a guiding line 12 configured to guide fuel ejected from the fuel pump 4 to the fuel property sensor 5; and a relief valve 21 configured to allow fuel to flow out of the guiding line 12 when a pressure of fuel in the guiding 12 becomes equal to or higher than a predetermined pressure.

### TECHNICAL FIELD

The present teachings relate to a sensor device.

### BACKGROUND ART

Patent Literature 1 (Japanese Patent Application Publication No. 2012-108030 A) discloses a sensor device that detects a concentration of alcohol contained in fuel. The sensor device includes a fuel pump that ejects the fuel and a fuel property sensor that detects a property of the fuel. The fuel ejected from the fuel pump is sent to the fuel property sensor, and the concentration of alcohol contained in the fuel is detected.

### SUMMARY OF INVENTION

### Technical Problem

The fuel ejected from the fuel pump is ejected under high pressure. When the high-pressure fuel is sent from the fuel pump to the fuel property sensor, the pressure of the fuel may cause a malfunction and/or a fuel leak in the fuel property sensor. It is therefore an object of the present teachings to provide a sensor device that makes it possible to suppress a malfunction and/or a fuel leak in a fuel property sensor.

### Solution to Technical Problem

A sensor device herein comprises: a fuel property sensor configured capable of detecting a property of fuel ejected from a fuel pump in a fuel tank; a guiding line configured to guide fuel ejected from the fuel pump to the fuel property sensor; and a relief valve configured to allow fuel to flow out of the guiding line when a pressure of fuel in the guiding line becomes equal to or higher than a predetermined pressure.

This configuration allows the pressure of fuel in the guiding line to be reduced by the outflow of fuel from the guiding line, thus making it possible to reduce the pressure of fuel that is sent to the fuel property sensor. This in turn makes it possible to suppress high pressure from acting on the fuel property sensor, thus making it possible to suppress a malfunction in the fuel property sensor. This also makes it possible to suppress fuel from leaking from the fuel property sensor.

### BRIEF DESCRIPTION OF DRAWINGS

FIG. 1 is a schematic view of a configuration of a sensor device according to an embodiment;

FIG. 2 is an enlarged cross-sectional view showing main components of the sensor device according to the embodiment;

FIG. 3 is a schematic view of a configuration of a sensor device according to another embodiment;

FIG. 4 is a schematic view of a configuration of a sensor device according to still another embodiment;

FIG. 5 is an enlarged cross-sectional view showing main components of a sensor device according to still another embodiment;

FIG. 6 is an enlarged cross-sectional view showing main components of a sensor device according to still another embodiment; and

FIG. 7 is an enlarged cross-sectional view showing main components of a sensor device according to still another embodiment.

### DESCRIPTION OF EMBODIMENTS

Some of the main features characteristic to below-described embodiments will herein be listed. It should be noted that the respective technical elements are independent of one another, and are useful solely or in combinations, The combinations thereof are not limited to those described in the claims as originally filed.

(Feature 1) A sensor device may further comprise a return line branching from the guiding line and configured to return fuel into the fuel tank. The relief valve may be arranged in the return line. According to this configuration, when the relief valve opens, the return line opens so that the fuel flows through the return line. The fuel flowing through the return line flows into the fuel tank. This causes the fuel ejected from the fuel pomp to be returned into the fuel tank, thus making it possible to reduce a pressure of fuel that is sent to the fuel property sensor.

(Feature 2) The sensor device may further comprise a transportation line branching from the guiding line. The transportation line is connected to a jet pump configured to introduce fuel in the fuel tank into a reserve cup arranged in the fuel tank, and send fuel from the guiding line to the jet pump. This makes it possible to reduce the pressure of fuel in the guiding line which communicates with the transportation line. This makes it possible to reduce the pressure of fuel that is sent to the fuel property sensor.

(Feature 3) The fuel property sensor may comprise a case forming a housing space capable of housing fuel whose property is to be detected, and a block portion formed integrally with the case. A flowing portion may be provided in the block portion, wherein the flowing portion communicates with the housing space, the guiding line and the return line, and is configured to allow fuel flowing from the guiding line to flow therethrough. The relief valve may be arranged in the flowing portion. This configuration allows the sensor device to be made in a small size.

(Feature 4) The sensor device may further comprise a set plate attached to the fuel tank. The fuel property sensor may be attached to the set plate. This configuration allows the sensor device to be made in a small size, as the set plate and the fuel property sensor are not positioned at a distance from each other.

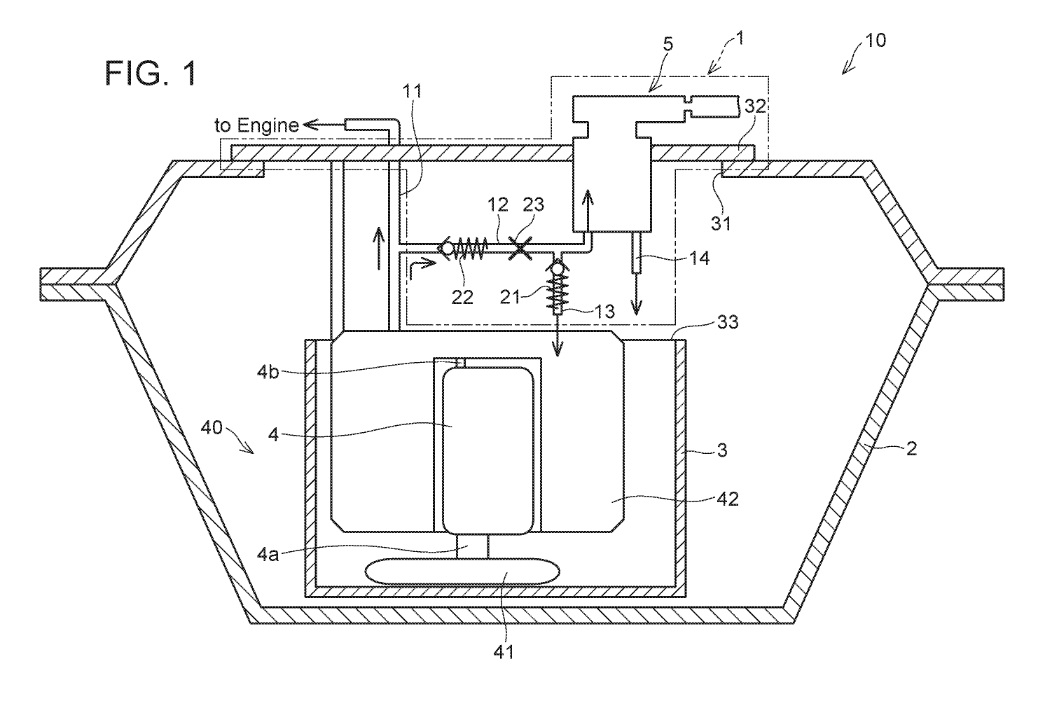

Embodiments will be described below with reference to the accompanying drawings. As shown in FIG. 1, a fuel supply unit 10 according to an embodiment comprises a fuel tank 2 that houses fuel and a sensor device 1 attached to the fuel tank 2. The fuel supply unit 10 supplies fuel to an engine of a vehicle. Further, the sensor device 1 detects a property of the fuel. More specifically, the sensor device 1 detects a concentration of alcohol contained in the fuel.

Fuel is stored in the fuel tank 2. The fuel contains gasoline and alcohol. Further, a pump unit 40 is disposed in the fuel tank 2. An opening 31 is provided in an upper part of the fuel tank 2.

The pump unit 40 comprises a reserve cup 3, a fuel pump 4, a suction filter 41, a high-pressure filter 42, and a supply line 11.

The reserve cup 3 is disposed in a bottom part of the fuel tank 2. The reserve cup 3 has an opening 33, and is disposed with the opening 33 facing upward. A portion of the fuel housed in the fuel tank 2 is stored in the reserve cup 3. Further, the fuel pump 4 is disposed in the reserve cup 3.

The fuel pump 4 sucks the fuel housed in the reserve cup 3, pressurizes the fuel thus sucked, and ejects the fuel thus pressurized. The fuel is ejected from the fuel pump 4 under high pressure. The fuel pump 4 is connected to an ECU (engine control unit; not illustrated) and driven under a control of the ECU.

The fuel pump 4 has a suction port 4a to which the suction filter 41 is attached. The suction filter 41 removes foreign matter from the fuel when the fuel pump 4 sucks the fuel. The fuel pump 4 has an ejection port 4b to which the high-pressure filter 42 is attached. The high-pressure filter 42 removes foreign matter from the fuel when the fuel pump 4 ejects the fuel.

One end of the supply line 11 is connected to the fuel pump 4 via the high-pressure filter 42. The other end of the supply line 11 is connected to the engine of the vehicle. The fuel ejected from the fuel pump 4 flows into the supply line 11 under high pressure. The fuel is sent to the engine of the vehicle through the supply line 11.

The sensor device 1 comprises a set plate 32 that is attached to the fuel tank 2 and a fuel property sensor 5 attached to the set plate 32. Further, the sensor device 1 comprises a guiding line 12, a return line 13, and a discharge line 14.

The set plate 32 is fixed to the upper part of the fuel tank 2 and closes the opening 31 of the fuel tank 2.

The fuel propel sensor 5 is fixed to the sot plate 32. The fuel property sensor 5 is a sensor configured to be capable of detecting a property of the fuel. More specifically, the fuel property sensor 5 is a sensor configured to detect a concentration of alcohol contained in the fuel. A usable example of the fuel property sensor 5 is a capacitive sensor configured to output a capacitance corresponding to a relative dielectric constant of the fuel as a signal corresponding to the concentration of alcohol.

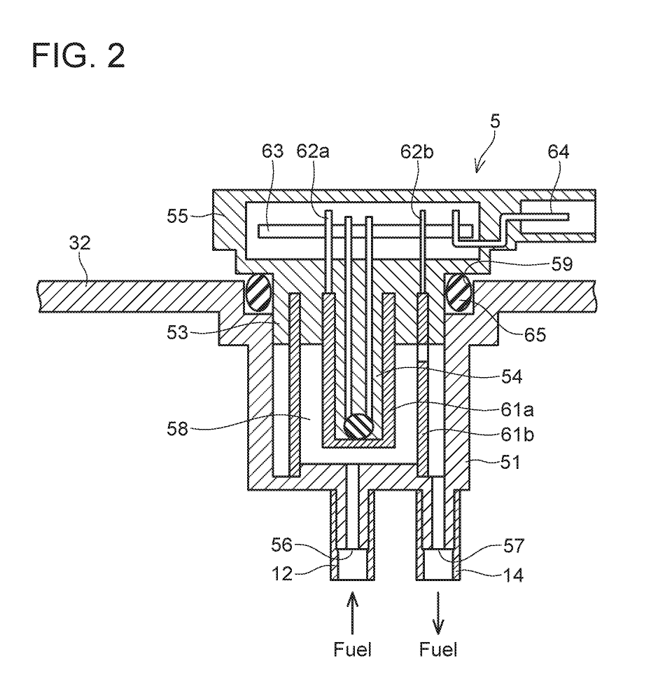

The fuel property sensor 5 is not limited to any particular configuration. However, in the present embodiment, as shown in FIG. 2, the fuel property sensor 5 comprises a lower case 51 and an upper case 55. The lower case 51 houses a pair of electrodes 61 (inner electrode 61a, outer electrode 61b), and the upper case 55 houses a circuit portion 63. The pair of the electrodes 61 (inner electrode 61a, outer electrode 61b) and the circuit portion 63 are electrically connected to each other via a pair of internal terminals 62a and 62b. Further, an external terminal 64 is electrically connected to the circuit portion 63. The circuit portion 63 processes electrical signals that are input from the internal terminals 62a and 62b, and outputs the electrical signals thus processed to an external circuit via the external terminal 64. The inner electrode 61a and the outer electrode 61b are in a shape of cylinders. The inner electrode 61a is housed on an inner side of the outer electrode 61b. The outer electrode 61b surrounds a periphery of the inner electrode 61a.

The lower case 51 is formed integrally with the set plate 32. The lower case 51 has an opening 59 formed therein. Further, the outer electrode 61b is disposed in the lower case 51. The outer electrode 61b is in contact with a bottom part of the lower case 51 and extends between the lower case 51 and the upper case 55 in an up and down direction. Further, the lower case 51 has a fuel inlet 56 and a fuel outlet 57 that are arranged in the bottom part thereof. The guiding line 12 is connected to the inlet 56 so that the fuel is introduced from the guiding line 12 into the lower case 51 via the inlet 56. The discharge line 14 is connected to the outlet 57 so that the fuel is discharged from the lower case 51 to the discharge line 14 via the outlet 57.

A lid portion 53 is fixed to the upper case 55. The lid portion 53 closes the opening 59 of the lower case 51. A sealing material 65 is disposed between the lower case 51 and the lid portion 53. The sealing material 65 tightly closes a gap between the upper case 51 and the lid portion 53. The lid portion 53 has a protruding portion 54 formed thereon. The protruding portion 54 extends downward. The inner electrode 61a is fixed to the protruding portion 54. Further, the outer electrode 61b is fixed to the lid portion 53 in such a way as to surround the inner electrode 61a. A surface of the inner electrode 61a faces a surface of the outer electrode 61b.

A space surrounded by the lower case 51, the lid portion 53, the inner electrode 61a, and the outer electrode 61b forms a housing space 58 capable of housing fuel whose property is to be detected. Fuel introduced into the lower case 51 from the inlet 56 flows through the housing space 58 to be discharged out of the lower case 51 from the outlet 57.

The pair of electrodes 61 (inner electrode 61a, outer electrode 61b) is a component configured to detect the capacitance of fuel. The pair of electrodes 61 (inner electrode 61a, outer electrode 61b) faces the housing space 58 and is configured to make contact with the fuel flowing through the housing space 58.

As shown in FIG. 1, the guiding line 12 has one end connected to the supply line 11 and the other end connected to the fuel property sensor 5. Fuel flowing through the supply line 11 flows into the guiding line 12. The guiding line 12 is configured to guide a portion of the fuel flowing through the supply line 11 to the fuel property sensor 5. This allows the fuel ejected from the fuel pump 4 to be guided to the fuel property sensor 5 by the guiding line 12. The fuel having flowed through the guiding line 12 is introduced into the fuel property sensor 5. The return line 13 branches from the guiding line 12.

Further, the guiding line 12 has a constricted portion 23. The constricted portion 23 is arranged on an upstream side of the part where the return line 13 branches. In the constricted portion 23, the guiding line 12 is narrow in cross-sectional area and makes it difficult for fuel to flow therein. The constricted portion 23 suppresses the flow of fuel from the supply line 11 toward the fuel property sensor 5. Further, the presence of the constricted portion 23 raises the pressure of fuel in the guiding line 12 on a supply line 11 side of the constricted portion 23. Further, the guiding line 12 is provided with a residual pressure holding valve 22. The residual pressure holding valve 22 is located on an upstream side of the constricted portion 23. The residual pressure holding valve 22 opens When the pressure of fuel in the guiding line 12 on the supply line 11 side of the residual pressure holding valve 22 becomes higher than a predetermined pressure, and closes when the pressure becomes equal to or lower than the predetermined pressure. Due to this, the residual pressure holding valve 22 holds the pressure of fuel on the supply line 11 side equal to or higher than the predetermined pressure. Therefore, the pressure of fuel flowing through the supply line 11 is maintained so that high-pressure fuel is sent to the engine of the vehicle.

The return line 13 has one end connected to the guiding line 12 and the other end opening toward an inside of the reserve cup 3. Fuel flowing through the guiding line 12 flows into the return line 13. The fuel having flowed through the return line 13 flows into the reserve cup 3. That is, fuel ejected from the reserve cup 3 by the fuel pump 4 passes through the guiding line 12, and is then returned into the reserve cup 3 by the return line 13.

A relief valve 21 is disposed in the return line 13. The relief valve 21 is provided on a downstream side of the residual pressure holding valve 22 and the constricted portion 23. As the relief valve 21, a valve can he used which includes a spring therein (not illustrated) and opens and closes according to a magnitude of a pressure acting thereon. The relief valve 21 opens when the pressure of fuel in the guiding line 12 becomes equal to or higher than the predetermined pressure. Since the return line 13 communicates with the guiding line 12, the pressure of fuel in the return line 13 becomes equal to or higher than the predetermined pressure when the pressure of fuel in the guiding line 12 becomes equal to or higher than the predetermined, pressure. Further, the predetermined or higher pressure acts on the relief valve 21. When the relief valve 21 opens, the return line 13 opens so that the fuel flows through the return line 13. Therefore, when the relief valve 21 opens, the fuel flows out of the guiding line 12 into the return line 13 so that the pressure of fuel in the guiding line 12 is reduced.

The discharge line 14 has one end connected to the fuel property sensor 5 and the other end opening toward an inside of the reserve cup 3. Fuel discharged from the fuel property sensor 5 flows into the discharge line 14. The fuel having flowed through the discharge line 14 flows into the reserve cup 3.

The following will describe an operation of the sensor device configured as above mentioned. In the sensor device 1, when the fuel pump 4 ejects the fuel in the reserve cup 3, the fuel thus ejected flows through the supply line 11 to be sent to the engine. Further, a portion of the fuel flowing through the supply line 11 flows into the guiding line 12, and flows through the guiding line 12 to be sent to the fuel property sensor 5. Then, the concentration of alcohol contained in the fuel is detected by the fuel property sensor 5. The relief valve 21 provided in the return line 13 shifts to an open state when the pressures of fuel in the guiding line 12 and the return line 13 become equal to or higher than the predetermined pressure when the fuel flows through the guiding line 12. When the relief valve 21 opens, the return line 13 opens so that the fuel flows through the return line 13 to be returned into the reserve cup 3.

As is evident from the foregoing descriptions, the sensor device 1 according to the embodiment allows the outflow of fuel from the guiding line 12 by causing the relief valve 21 to open when the pressure of fuel in the guiding line 12 becomes equal to or higher than the predetermined pressure. This makes it possible to reduce the pressure of fuel in the guiding line 12, thus making it possible to reduce the pressure of fuel that is sent to the fuel property sensor 5. This in turn makes it possible to suppress high pressure from acting on the fuel property sensor 5, thus making it possible to suppress a malfunction in the fuel property sensor 5. This also makes it possible to suppress fuel from leaking from the fuel property sensor 5.

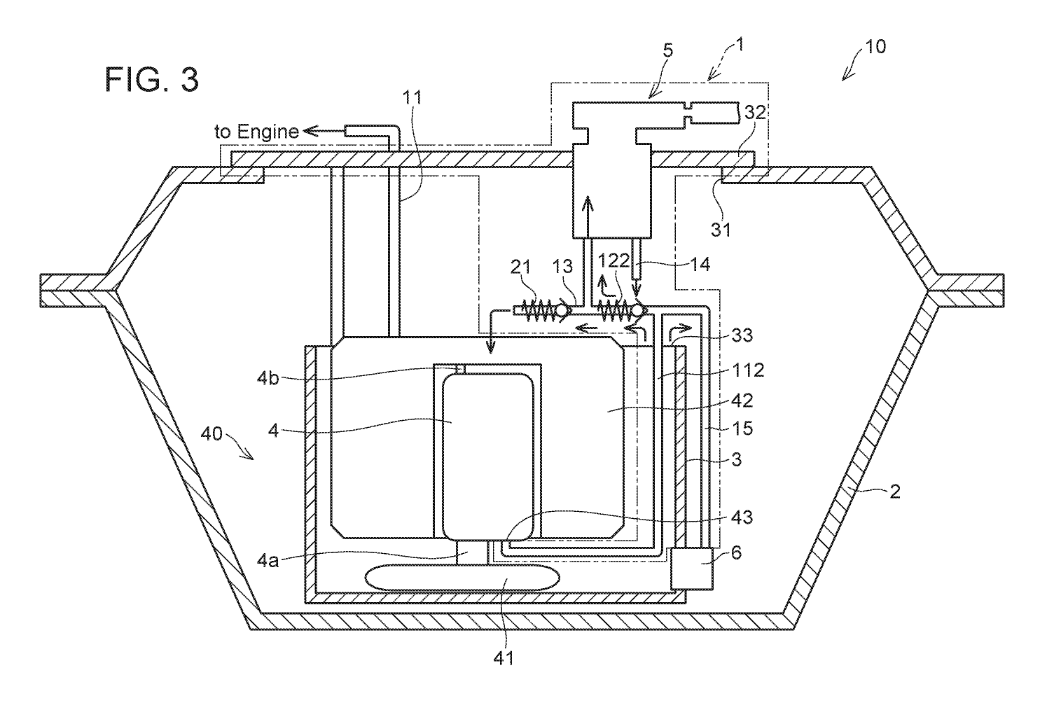

While one embodiment has been described above, a specific aspect is not limited to the embodiment. For example, while the guiding line 12 branched from the supply line 11 in the embodiment, a configuration of the guiding line is not limited to the embodiment in another embodiment, as shown in FIG. 3, a guiding line 112 is connected to the fuel pump 4. Components shown in FIG. 3 that are identical to those shown in FIG. 1 are given the same signs, and descriptions thereof will be omitted. The fuel pump 4 includes a vapor jet 43 configured to allow vapor generated in the fuel pump 4 to be discharged out of the fuel pump 4. The guiding line 112 has one end connected to the vapor jet 43 of the fuel pump 4 and the other end connected to the fuel property sensor 5. Fuel pressurized by the fuel pump 4 is ejected from the vapor jet 43 to the guiding line 112 together with the vapor. The fuel and vapor thus ejected are guided to the fuel property sensor 5 by the guiding line 112 and flow into the fuel property sensor 5.

Further, a fuel supply unit 10 shown in FIG. 3 includes a jet pump 6 configured to introduce fuel in the fuel tank 2 into the reserve cup 3. The sensor device 1 includes a transportation line 15 branching from the guiding line 112 and configured to send fuel to the jet pump 6. The jet pump 6 is attached to a bottom part of the reserve cup 3. The jet pump 6 is configured to introduce fuel outside the reserve cup 3 into the reserve cup 3 by utilizing a velocity of fuel ejected from the vapor jet 43. The transportation line 15 has one end connected to the guiding line 112 and the other end connected to the jet pump 6. A portion of the fuel and vapor flowing through the guiding line 112 flows into the transportation line 15. The fuel having Rowed through the transportation line 15 is introduced into the reserve cup 3 by the jet pump 6. The guiding line 112 is provided with a residual pressure holding valve 122. The residual pressure holding valve 122 is provided on a downstream side of the part where the transportation line 15 branches from the guiding line 112. The residual pressure holding valve 122 opens When the pressure of fuel in the guiding line 112 on an upstream side of the residual pressure holding valve 122 becomes higher than a predetermined pressure, and closes when the pressure becomes equal to or lower than the predetermined pressure. Further, the return line 13 branches from the guiding line 112.

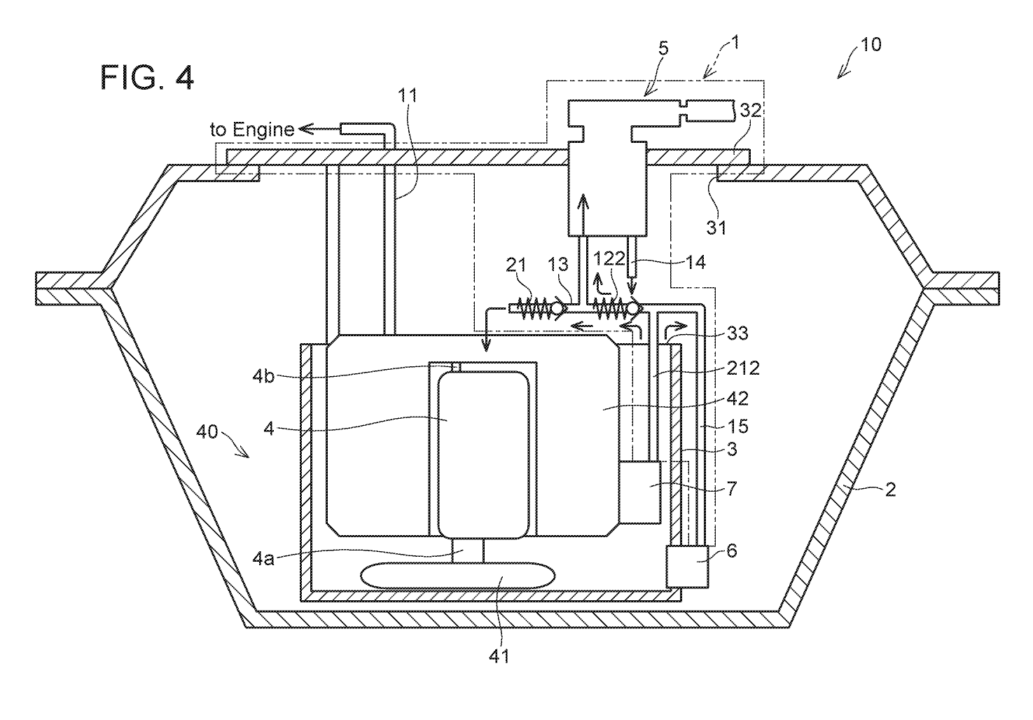

Further, while the guiding line 112 was connected to the vapor jet 43 in the embodiment, a configuration of the guiding line is not limited to the embodiment. In still another embodiment, as shown in FIG. 4, a guiding line 212 is connected to a pressure regulator 7. Components shown in FIG, 4 that are identical to those shown in FIG. 3 are given the same signs, and descriptions thereof will be omitted. The pressure regulator 7 adjusts the pressure of fuel ejected from the fuel pump 4. The fuel pressurized by the fuel pump 4 flows into the pressure regulator 7. The pressure regulator 7 adjusts the pressure by ejecting into the reserve cup 3 a portion of the fuel having flowed into the pressure regulator 7.

Further, while the relief valve 21 is provided in the return line 13 in the embodiment, the relief valve 21 may be provided in the guiding line 12, 121, or 212 without being limited to this configuration. In this case, when the relief valve 21 opens, fuel is released from the guiding line 12, 121, or 212 to the outside.

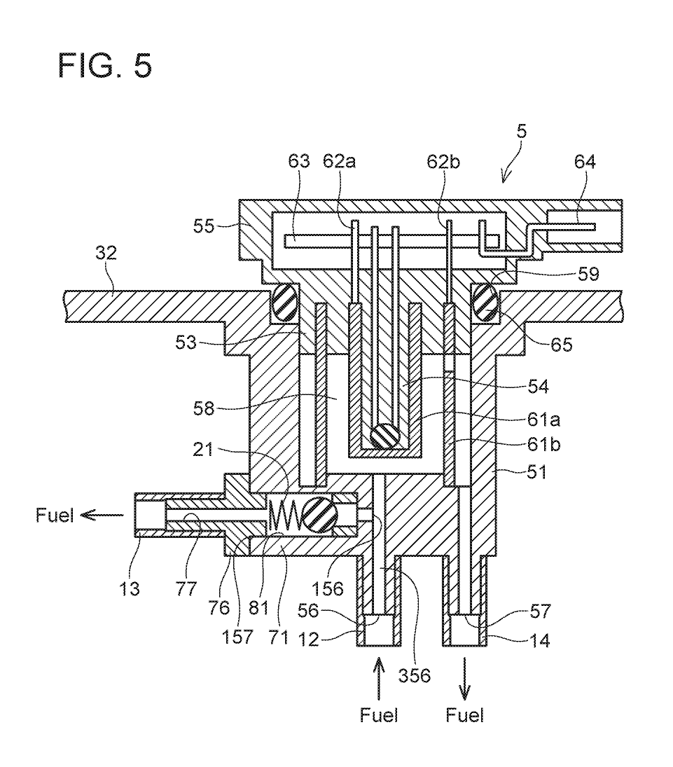

Further, without being limited in configuration to the embodiment, the relief valve 21 may be formed integrally with the fuel property sensor 5. In this case, for example as shown in FIG. 5, the fuel property sensor 5 includes a block portion 71 formed integrally with the lower case 51. Components shown, in FIG, 5 that are identical to those shown in FIG. 2 are given the same signs, and descriptions thereof will be omitted. The block portion 71 is arranged in a lower part of the lower case 51. Provided in the block portion 71 is a flowing portion 81 configured to allow flow of fuel therein. Further, the block portion 71 has an inlet 156 and an outlet 157 formed therein. The inlet 156 of the block portion 71 communicates with a flow passage 356 connecting the inlet 56 of the lower case 51 and the housing space 58. A connector 76 is attached to the outlet 157 of the block portion 71. The outlet 157 of the block portion 71 is connected to the return line 13 via the connector 76.

The flowing portion 81 is formed by hollowing out an inside portion from the block portion 71. The flowing portion 81 communicates with the return line 13 via the outlet 157 and the connector 76. Further, the flowing portion 81 communicates with the housing space 58 and the guiding line 12 via the inlet 156 and the flow passage 356. Further, the relief valve 21 is disposed in the flowing portion 81, and when the relief valve 21 opens, the flowing portion 81 opens. The connector 76 has a connector flow passage 77. The connector flow passage 77 communicates with the flowing portion 81 of the block portion 71 and the inside of the return line 13. The fuel having flowed into the flowing portion 81 from the inlet 156 of the block portion 71 is discharged to the connector flow passage 77 from the outlet 157 and flows out to the return line 13.

In such a configuration, fuel having flowed through the guiding line 12 is introduced into the housing space 58 via the flow passage 356. Further, fuel flowing through the flow passage 356 is introduced into the flowing portion 81 via the inlet 156. When the pressure of fuel in the guiding line 12 rises, the pressure of fuel in the flowing portion 81 rises as well. Moreover, when the pressure of fuel in the guiding line 12 becomes equal to or higher than a predetermined pressure, the relief valve 21 in the flowing portion 81 opens. When the relief valve 21 opens, the flowing portion 81 opens so that the fuel flows from the flowing portion 81 to the return line 13 through the connector 76. This causes the fuel to flow out of the guiding line 12 into the return line 13 so that the pressure of fuel in the guiding line 12 is reduced.

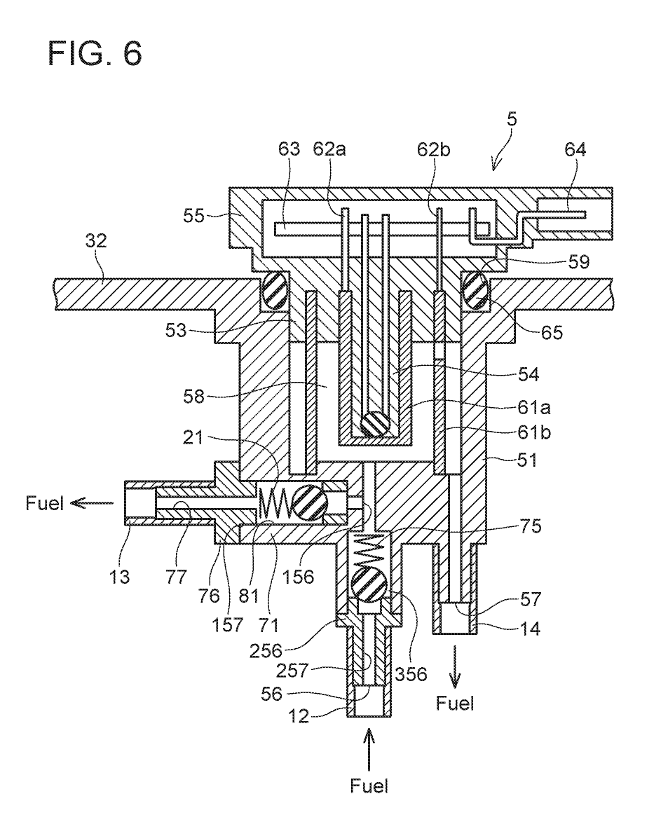

Further, the flow passage 356 is not limited in configuration to the embodiment. As shown in FIG. 6, a residual pressure holding valve 75 may be disposed in the flow passage 356. Components shown in FIG. 6 that are identical to those shown in FIG. 5 are given the same signs, and descriptions thereof will be omitted. The flow passage 356 has a portion that is larger in cross-sectional area than any other portion of the flow passage 356. The residual pressure holding valve 75 is disposed in the portion of the flow passage 356 that is large in cross-sectional area. The residual pressure holding valve 75 opens when the guiding line 12 becomes equal to or higher than a predetermined pressure. When the residual pressure holding valve 75 opens, the flow passage 356 opens so that the fuel flows therethrough. Further, a connector 256 is attached to the flow passage 356. The connector 256 connects the guiding line 12 to the lower case 51. The connector 256 has a connector flow passage 257. The connector flow passage 257 communicates with the flow passage 356 and the inside of the guiding line 12. The fuel flows from the guiding line 12 into the flow passage 356 through the connector flow passage 257.

Further, while, in the embodiment, the return line 13 and the discharge line 14 are configured to send fuel into the reserve cup 3, the fuel does not necessarily have to be sent into the reserve cup 3, as long as the fuel can he sent into the fuel tank 2. For example, the return line 13 and the discharge line 14 may be configured to send fuel to the inside of the fuel tank 2 and outside of the reserve cup 3.

Further, while, in the embodiment, the fuel property sensor 5 is configured to detect the concentration of alcohol contained in fuel, a property of the fuel that is detected by the fuel property sensor 5 is not limited to the concentration of alcohol contained in the fuel. For example, the fuel property sensor 5 may be configured to detect a property of fuel such as a degree of deterioration of the fuel a liquid level (liquid surface height) of the fuel, or a temperature of the fuel.

Further, the fuel property sensor 5 is not limited in configuration to the embodiment and may be configured in various ways, for example, to have a plate-shaped electrode or to have a comb-shaped electrode fixed on a substrate.

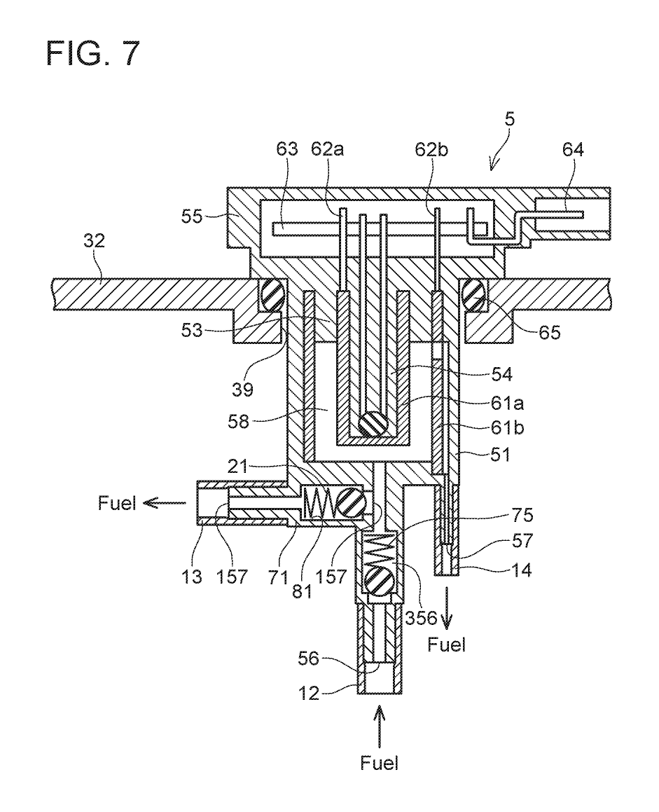

Further, while the lower case 51 and the upper case 55 were formed separately from each other in the embodiment, the lower case 51 and the upper case 55 are not limited to this configuration. In a fuel property sensor 5 according to still another embodiment, as shown in FIG. 7, the lower case 51 and the upper case 55 are formed integrally with each other. Further, while the lower case 51 and the set plate 32 were formed integrally with each other in the embodiment, the lower case 51 and the set plate 32 are not limited to this configuration. In the fuel property sensor 5 according to still another embodiment, as shown in FIG. 7, the lower case 51 and the set plate 32 are formed separately from each other. The lower case 51 is inserted. in an opening 39 formed in the set plate 32. This causes the fuel property sensor 5 to be attached to the set plate 32.

Specific examples of the present teachings have been described in detail, however, these are mere exemplary indications and thus do not limit the scope of the claims. The art described in the claims includes modifications and variations of the specific examples presented above. Technical features described in the description and the drawings may technically be useful alone or in various combinations, and are not limited to the combinations as originally claimed. Further, the art described in the description and the drawings may concurrently achieve a plurality of aims, and technical significance thereof resides in achieving any one of such aims.

### Reference Signs List

1: Sensor device
2: Fuel tank
3: Reserve cup
4: Fuel pump
4a: Suction port
4b: Ejection port
5: Fuel property sensor
6: Jet pump
7: Pressure regulator
11: Supply line
12: Guiding line
13: Return line
14: Discharge line
15: Transportation line
21: Relief valve
22: Residual pressure holding valve
23: Constricted portion
31: Opening
32: Set plate
33: Opening
39: Opening
40: Pump unit
41: Suction filter
42: High-pressure filter
43: Vapor jet
51: Lower case
53: Lid portion
54: Protruding portion
55: Upper case
56: Inlet
57: Outlet
58: Housing space
59: Opening
61: Electrodes
62: Internal terminals
63: Circuit portion
64: External terminal
65: Sealing material
71; Block portion
76: Connector
77: Connector flow passage
81: Flowing portion
156: Inlet
157: Outlet
256: Connector
257: Connector flow passage
356: Flow passage

1. A sensor device comprising:
a fuel property sensor configured capable of detecting a property of fuel ejected from a fuel pump in a fuel tank;
a guiding line configured to guide fuel ejected from the fuel pump to the fuel property sensor; and
a relief valve configured to allow fuel to flow out of the guiding line when a pressure of fuel in the guiding line becomes equal to or higher than a predetermined pressure.

2. The sensor device according to claim 1, further comprising
a return line branching from the guiding line and configured to return fuel into the fuel tank,
wherein
the relief valve is arranged in the return line.

3. The sensor device according to claim 1, further comprising
a transportation line branching from the guiding line,
wherein
the transportation line is connected to a jet pump configured to introduce fuel in the fuel tank into a reserve cup arranged in the fuel tank, and send fuel from the guiding line to the jet pump.

4. The sensor device according to claim 1, wherein
the fuel property sensor comprises a case forming a housing space capable of housing fuel whose property is to be detected, and a block portion formed integrally with the case,
a flowing portion is provided in the block portion, wherein the flowing portion communicates with the housing space, the guiding line and the return line, and is configured to allow fuel flowing from the guiding line to flow therethrough, and
the relief valve is arranged in the flowing portion.

5. The sensor device according to claim 1, further comprising
a set plate attached to the fuel tank,
wherein
the fuel property sensor is attached to the set plate.
